(19) United States
(12) Patent Application Publication
(10) Pub. No.: US 2021/0403392 A1
(43) Pub. Date: Dec. 30, 2021

(54) MICROALGAE-BASED SOIL NON-ELECTRIC INOCULATING SYSTEM AND METHODS OF USE

(71) Applicant: MYLAND COMPANY, LLC, Phoenix, AZ (US)

(72) Inventors: Dane Hague, Phoenix, AZ (US); Andrew D. Ayers, Phoenix, AZ (US); Philip Risser, Phoenix, AZ (US)

(21) Appl. No.: 17/361,738

(22) Filed: Jun. 29, 2021

Related U.S. Application Data
(60) Provisional application No. 63/045,630, filed on Jun. 29, 2020.

(51) Int. Cl. C05F 17/90; C12M 1/12; C12M 1/26; C12M 1/00

(52) U.S. Cl. CPC C05F 17/989; C12M 45/00; C12M 33/14; C12M 37/00

(57) ABSTRACT

Some embodiments include a microalgae culturing system including a bioreactor adapted to propagate microalgae in a culture solution using in combination at least one of natural and artificial light, and at least one nutrient including at least a carbon source, where the microalgae are freely suspended in and form part of the culture solution. A microalgae feed source is coupled to the bioreactor and a first controller between a water conditioning assembly and the bioreactor. The water conditioning assembly is coupled as an input of supply water to the bioreactor, and configured to condition the supply water to a specified purity that enables substantially unhindered growth of the microalgae in the culture solution to a specified concentration, and the first controller is configured to control supply of the microalgae feed source to the bioreactor.

### CROSS REFERENCE TO RELATED APPLICATIONS

This application claims priority to and the benefit of U.S. Provisional Patent Application No. 63/045,630, filed Jun. 29, 2020, the entire contents of which are incorporated by reference herein.

### BACKGROUND

Microbes in soil have many beneficial effects. In addition, microorganisms, such as algae, have the ability to adapt to their environment. For instance, algae found in soil in the Southwestern deserts have adapted to elevated temperatures, alkaline pH levels, and periods of desiccation, while algae in northern climates have adapted to much lower temperatures, freeze-thaw cycles, higher soil moisture levels, and more acidic soil pH levels, etc.

Endemic algae fill a niche in the field ecosystem. Within the soil ecosystem, a symbiosis with other organisms has developed resulting in a biochemical environment where compounds produced by the endemic algae may augment the growth of other desirable microbes and depress the growth of undesirable or non-beneficial organisms. For example, algae produce biochemicals such as amino acids, hormones, peptides, and fatty acids that augment the growth of other beneficial microorganisms. These beneficial biochemicals directly help crop plants. The beneficial microorganisms produce biochemicals that the algae and crop can utilize to grow (e.g., sugars and vitamins). At the same time, algae may produce compounds that are antibacterial, antifungal, algicidal, and/or antiprotozoal which prevent the growth of unwanted microbes in soil and surface waters.

When soil algae die, cellular biochemicals are released which can directly feed the soil biome and any crop plants growing in the soil. These biochemicals are large molecules (e.g., such as proteins, fats, dyes, peptides, nucleic acids, etc.), some or all of which can be absorbed by the crop plant, resulting in crops with greater nutritional value. If live, foreign algae are introduced into the soil, the ecosystem is forced to rebalance. This imbalance can lead to the production of one or more unwanted biochemicals (such as a toxin), or the absence of an important biochemical which may be required by the crop plant.

When algae are introduced to the soil, the algae utilize the available root exudates and sugars from the decomposition of plant debris as a food source and multiply exponentially increasing the total microbial population. As some of the algal cells are consumed by the other members of the microbial community the population and the metabolic activity in the soil increases, resulting in greater CO₂ production. This is particularly true for live algae whose metabolic activities continue after introduction to the soil. This CO₂ production lowers the pH of the soil resulting in the dissolution of calcium and magnesium carbonate bonds, thereby opening the soil for greater root penetration and increased water and fertilizer movement. The increased water movement carries more salts out of the root zone, thereby reducing the osmolarity within the root zone, and increasing the bioavailability of macro and micronutrients to the crop. The lower pH also frees up bound potassium and phosphorus making it available to the plants. Algae excrete extracellular phosphatases almost immediately upon the onset of phosphorus limited conditions. These compounds release the phosphates from soil particles and make them available to the plants. Green algae also produce polysaccharides which hold onto water until it is needed.

Conventional methods of introducing humus to soil generally requires tilling-in of organic matter (compost, various plant cuttings, manures, etc.), which can best be performed when a field is between plantings. Humus aids in the formation of natural iron chelates (fulvic acids-Fe), which prevents soil from being blocked by calcium and magnesium carbonates, thus avoiding chlorosis problems induced by low bioavailability of these nutrients. Chlorosis is the reduction in the green color of plants due to a reduction in the amount of chlorophyll in the leaves brought on by a lack of bioavailable macro and micronutrients such as nitrogen (N), magnesium (Mg), calcium (Ca), and iron (Fe), even when these nutrients are present in the soils.

Ion exchange capacity is a quantitative means for describing the binding of fertilizer elements to soil particles for storage and release. Humus ion exchange capacity (e.g., 400 to 600 meq/100 g) is 5 to 10 times higher than that of clays (e.g., 50 to 150 meq/100 g). It is this capacity which allows the retention of fertilizers within the soil for use by the plants as needed. As plants utilize the nitrogen (N), phosphorus (P), and potassium (K) in the soil, the stored elements are released from the humus as needed. By combining with humic substances, copper and other trace elements become less toxic and more readily available to the plants.

However, an inexpensive system and method for cultivating and propagating endemic algae and delivering it to a target geographical location does not currently exist.

### SUMMARY

Some embodiments of the disclosure are directed to systems and methods of adding algae (any reference to algae is also a reference to microalgae and vice versa as used herein) along with one or more other living organisms described herein to topsoil. Fertilizers are more effective if combined with microalgae. In some embodiments, the system is configured to enable substantially constant and/or periodic addition of algae to maintain sufficient number of cells to capture the majority of the organic materials produced by the plant roots and other microbes resulting in a desirable buildup of organic matter (humus) within the soil. In some embodiments, the system is configured to deliver a sufficient amount of algae to hold water and nutrients which can be released to the plants as needed. In some embodiments, the algae cells process fertilizers by breaking down certain molecules into more bioavailable forms that plants can more readily use. The nutrients are then more efficiently and completely absorbable by the root system of the plants. For example, ammonium nitrate, an excellent source of nitrogen, is one of the most common bulk fertilizers used to grow crops. While plants can immediately absorb the nitrate in this fertilizer, the ammonium component is less accessible to the plant. In some embodiments, the system is configured to deliver algae cells to the topsoil and/or fertilizer to absorb the ammonium, naturally convert it to nitrogenous biochemicals, and upon their death, release these valuable biochemicals to the plant for easy consumption. Additionally, the nutrients from fertilizers can bind to the microalgae cells or their organic remains and are less likely to be lost in run-off water during rains or irrigation. Upon their death, the algae can also feed bacteria in the soil, which can convert the ammonium ion into nitrate ions.

Some embodiments of the system are directed to cultivating algae to produce growth regulators (e.g., gibberellic acid) that improve salt tolerance, induce seed germination and increase plant growth rate and fruit production. Current commercially available artificial or concentrated growth regulators are expensive, especially when applied in substantial amounts, making it impractical for growers to use in bulk.

Some embodiments are directed to cultivating algae and delivering the algae to topsoil to play a role in controlling agricultural pests by providing a system to produce antibiotics and antifungal compounds, and for feeding the beneficial microbes in the soil which produce other pest fighting compounds. These compounds give the plants the ability to prevent the invasion of pathogenic species. Disease and pests are also resisted due to the improved vigor of the plants.

As discussed above, the system is configured to deliver live algae cells that function as a catalyst to tap and utilize all of the benefits available from standard fertilizers; and also, to provide a natural supply of essential compounds and phytochemicals, while supporting the overall efficacy of the growing environment according to some embodiments. In some embodiments, these potent attributes work in concert to stimulate plants to grow heartier and more quickly; and to consistently produce a more abundant, higher quality and more nutrient rich end-product such as a crop. The benefits from an additive of microalgae cells are available when the algae cells that are delivered to the soil are in healthy living form and in great concentration, therefore, in some embodiments, the system is configured to deliver healthy living algae to a system outlet (also referred to as an algae outlet) for subsequent distribution to a topsoil environment. In some embodiments, the selection and formulation of the algae additive is critical to its overall impact to a specific environment and/or geographical location. Some embodiments of the disclosure are directed to a microalgae additive system and method that is simple to manage, and offers breakthrough potential in agricultural production. Unexpectedly, the most depleted soils such as arid soils that have significant salt and caliche buildup with minimal organic matter have been rejuvenated to the point of producing significantly improved crop yield using the systems and methods described herein. Further, in some embodiments, methods include sampling endemic algae from the target location, cultivating the sample in one or more bioreactors, and implementing aspects of the system for propagation of the endemic algae and delivery to an agricultural production area. By using the systems and methods described herein, in some embodiments, there is a higher survival rate for the algae, and a greater and faster impact on soil health where the algae produced by the system is applied.

Some embodiments include a culturing system comprising a bioreactor adapted to propagate microalgae in a culture solution using natural light and/or artificial lights, and at least one nutrient supply comprising a fertilizer solution. In some embodiments, the algae are freely suspended in and/or form part of the culture solution. In some embodiments, the at least one nutrient supply includes an algae nutrient supply coupled to the bioreactor and a water conditioning assembly and the bioreactor. In some embodiments, the water conditioning assembly is coupled as an input of supply water to the bioreactor, and configured to condition the supply water to a specified purity that enables substantially unhindered growth of the algae in the culture solution to a specified concentration. In some embodiments, the water conditioning assembly includes one or more filter and/or one or more sources of ultraviolet (UV) light.

Some embodiments include at least one pressurized gas supply system (e.g., a blower, one or more filters) coupled to the bioreactor, where the at least one pressurized gas supply system is configured to generate gas bubbles to at least partially aerate and/or agitate the culture solution. In some embodiments, the gas bubbles include one or more of air, CO₂, N₂, and/or O₂. Some embodiments further comprise at least one water reservoir or tank providing or coupled to the fluid inlet.

In some embodiments, the at least one nutrient supply comprises a fertilizer, a macro-nutrient, a micro-nutrient, and at least one or more microalgae species. In some embodiments, the macro-nutrient is selected from the group consisting of phosphorus, nitrogen, carbon, silicon, calcium salt, magnesium salt, sodium salt, potassium salt, and sulfur; and the one or more micronutrients is selected from the group consisting of manganese, copper, zinc, cobalt, molybdenum, vitamins and trace elements. Further, in some embodiments, the micro-nutrient comprises one or more vitamins and/or minerals added to the inlet fluid.

Some embodiments comprise a telemetry system configured for a remote monitoring and/or controlling operation of one or more of a first controller, a second controller, the bioreactor, and at least one component or assembly of the water conditioning assembly.

In some further embodiments, the microalgae feed source comprises one or more of a first algae type, a second algae type, bacteria, and fungi. Some embodiments further comprise a microorganism mixer configured to blend one or more of algae, bacteria, and fungi, with any of the culture solution exiting the bioreactor.

Some embodiments include a method comprising preparing one or more microbe-containing samples from at least one location of a current or planned plant growth area, and preparing at least one cultured sample by culturing microbes from the sample. Further, some embodiments include selecting at least one target species of microbe from the at least one cultured sample and propagating the at least one selected target species of microbe to increase the concentration of the at least one target species of microbe in the at least one cultured sample. Some embodiments include providing a bioreactor adapted to propagate the at least one selected target species in a culture solution. In some embodiments, the method includes placing the culture solution into the bioreactor, where the at least one selected target species is freely suspended in and forming part of the culture solution.

Some embodiments include delivering at least a portion of the at least one target species of microbe to at least a portion of the at least one location (e.g., within 50 miles in some embodiments) where the one or more microbe containing samples was obtained, where at least a portion of the at least one target species of microbe being delivered comprises at least one live microbe (e.g., algae) from the one or more bioreactors. In some embodiments, the at least one live microbe is selected to be a well-adapted endemic species. In some embodiments, the at least one live microbe is an endemic species of algae. In some further embodiments, the at least one live microbe is a live species selected to restore a normal soil flora mix of a cropland. In some embodiments, the live species of algae is selected for its specific desired properties for improving the soil in the delivery location.

Some embodiments include a method comprising sampling the algal flora from an agricultural location, and selecting at least one desired algae species for propagation, where the at least one desired algae species is present in the agricultural location as an initial concentration. Some embodiments include propagating the at least one desired algae species in at least one bioreactor, and delivering the at least one desired species to the agricultural location to increase the concentration of the algae species to a concentration greater than the initial concentration.

In some embodiments, the at least one bioreactor is adapted to propagate at least one desired species in a culture solution using natural and/or artificial light, and at least one nutrient comprising at least one carbon source, where at least one desired species are freely suspended in and form part of the culture solution.

In some embodiments, at least one nutrient supply is coupled to the at least one bioreactor and/or at least one metering pump for controlling flow. In some embodiments, the water conditioning assembly is coupled as an input of supply water to the at least one bioreactor to condition the supply water to a specified purity that enables substantially unhindered growth of the microalgae in the culture solution to a specified concentration. In some embodiments, a specified purity includes the killing and/or removal of microorganisms harmful to the algae propagating in the bioreactor. Further, in some embodiments, the metering pump is configured to control supply of the algae nutrient supply to the at least one bioreactor.

### DESCRIPTION OF THE DRAWINGS

FIG. 1 depicts a non-limiting example of the microalgae-based soil inoculating system according to some embodiments.

FIG. 2 illustrates a solar power system configured to deliver electrical energy to one or more system components according to some embodiments.

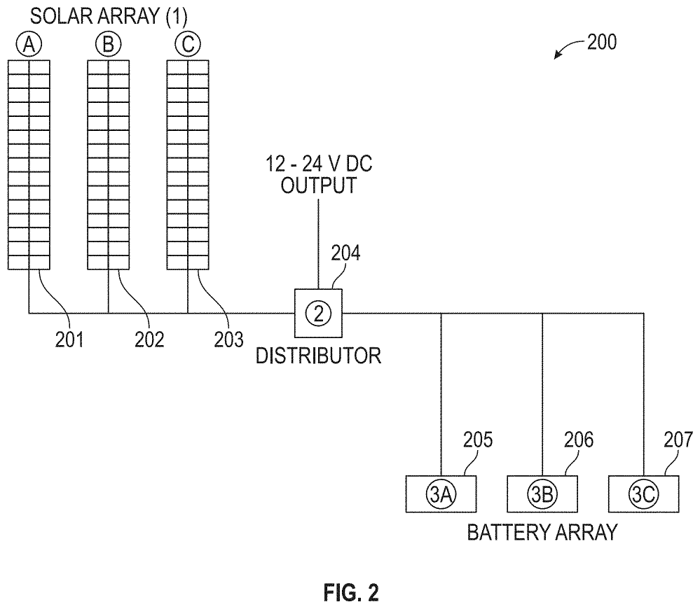

FIG. 3 depicts an arrangement of the system that includes a plurality of bioreactors according to some embodiments.

FIG. 4 illustrates the arrangement for the 1.77 acre plot described in Example 3 according to some embodiments.

FIG. 5 illustrates the yield increase results from Example 3 according to some embodiments.

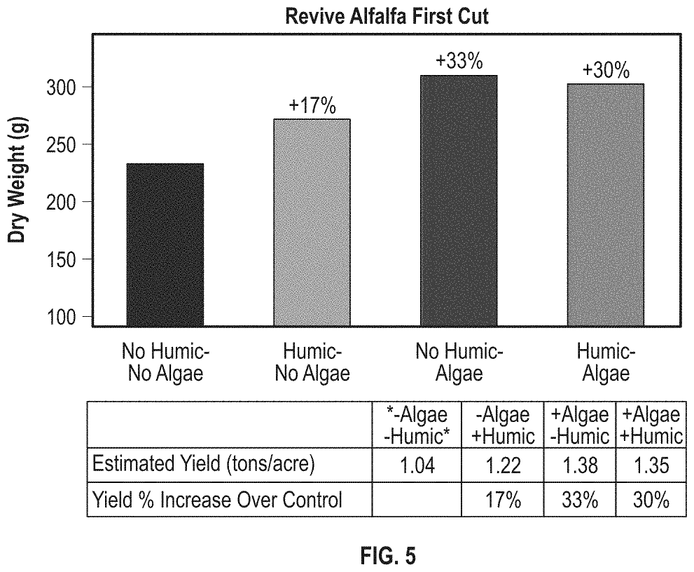

FIG. 6 is a chart showing the increase in microbial biomass results from Example 3.

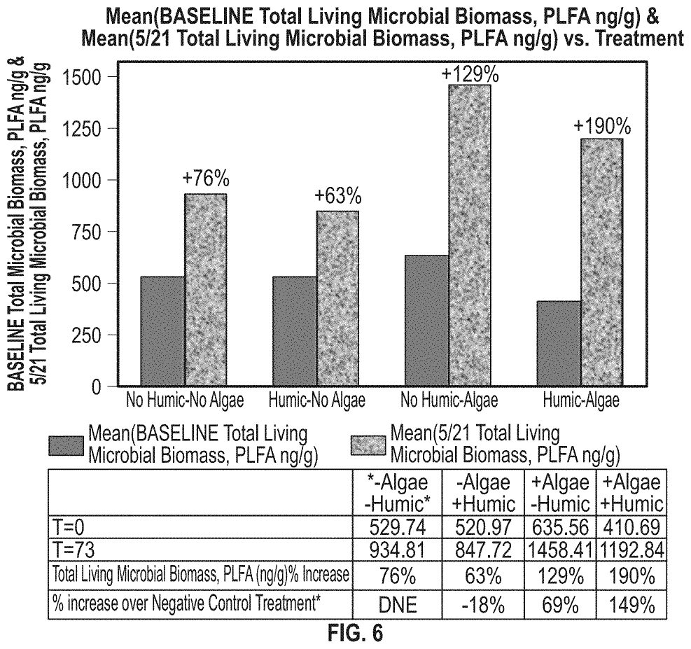

### DETAILED DESCRIPTION

Before any embodiments of the system are explained in detail, it is to be understood that the system is not limited in its application to the details of construction and the arrangement of components set forth in the following description or illustrated in the following drawings. The system is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the system. Various modifications to the illustrated embodiment will be readily apparent to those skilled in the art, and the generic principles herein can be applied to other embodiments and applications without departing from embodiments of the system. Thus, embodiments of the system are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the system. Skilled artisans will recognize the examples provided herein have many useful alternatives that fall within the scope of embodiments of the system.

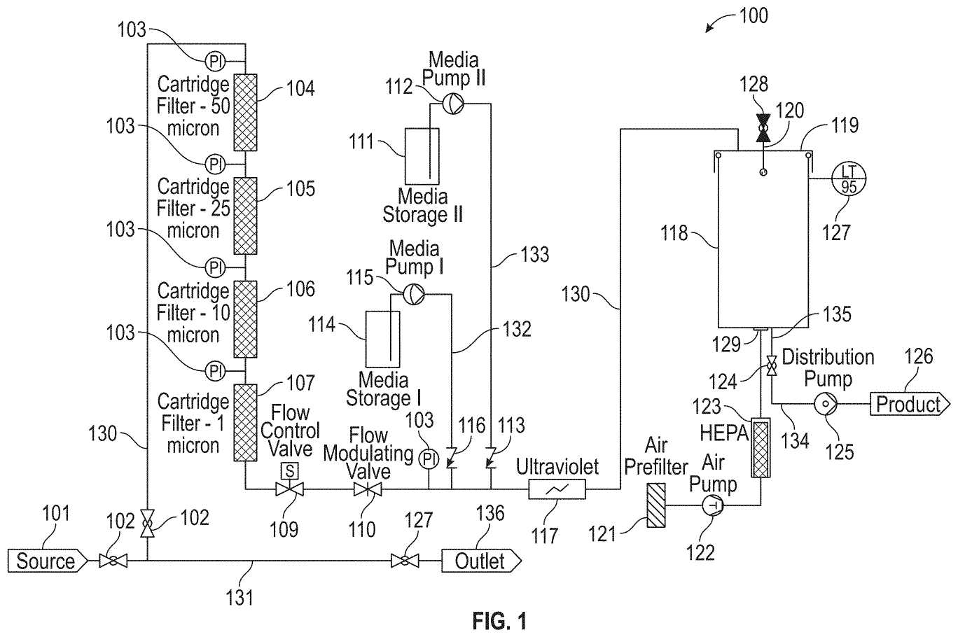

FIG. 1 depicts a non-limiting example of some aspects of a microalgae-based soil inoculating system 100 according to some embodiments. In some embodiments, the system 100 is configured to draw fluid (e.g., water) from a fluid source inlet 101. In some embodiments, the system includes one or more inlet isolation valves 102 configured to isolate the fluid source inlet 101 from one or more components of the system. In some embodiments, the fluid source inlet 101 includes one or more of a fluid storage tank, a fluid supply line, a fluid well, and/or any other source of fluid suitable for irrigation purposes and/or algae cultivation (not shown) and may also include a pressurization system (e.g., gravity, one or more pumps; not shown) to overcome any resistance. In some embodiments, an outlet isolation valve 127 is closed while supplying fluid to the system through isolation valves 102 to ensure adequate fluid pressure is present to force fluid through an inlet conduit 130. In some embodiments, when sufficient fluid pressure is present the system is configured to enable fluid flow through both the inlet conduit 130 and the fluid supply conduit 131 simultaneously. In some embodiments, the fluid source outlet 136 of the fluid supply conduit 131 is connected to a conventional irrigation system. In some embodiments, the algae outlet 126 is connected to the same and/or a different irrigation system, where the outlet pump 125 is configured to overcome the pressure in fluid supply conduit 131 and/or to introduce an irrigation and algae mixture to a target location.

In some embodiments, the inlet conduit 130 includes one or more pressure gauges 103 configured to monitor inlet pressure at various stages. In some embodiments, inlet fluid is initially passed through one or more inlet fluid filters 104-107 (e.g., mechanical filters, screens, cartridge filters, centrifugal filters, and/or any conventional filter) configured to remove particulates from the inlet fluid. In some embodiments, the one or more filters are configured to remove particulates in the range of 1-50 microns. In some embodiments, the inlet conduit 130 comprises a series of inlet fluid filters 104-107, where each filter is configured to remove a progressively smaller size of particulate. As shown in the non-limiting example system 100 of FIG. 1, some embodiments include 50, 25, 10, and 1 micron filters placed in series to remove particulate from incoming fluid.

The inlet conduit 130 further includes one or more flow control valves 109 and flow modulating valves 110 according to some embodiments. In some embodiments, the flow control valve 109 is configured to control an inlet pressure into the system 100. In some embodiments, the flow modulating valve 110 is configured to control a flowrate through the system 100. In some embodiments, either valve 109 or 110 can be used to control pressure and/or flowrate through the system 100. In some embodiments, either valve 109 or 110 can be used to isolate at least a portion of the system 100.

In some embodiments, the system is configured to supply nutrients to the inlet conduit 130. In some embodiments, the nutrients include one or more types of feed and nutrients such as those described herein which promote the growth of algae in one or more bioreactors 118. In some embodiments, one or more check valves 113, 116 at least partially isolate one or more nutrient supply conduits 132, 133 from the pressure in the inlet conduit 130 and/or prevent the flow of fluid from inlet conduit 130 to the one or more nutrient source suppliers 111, 114. When nutrient delivery to the one or more bioreactors 118 is desired, in some embodiments, one or more pumps 112, 115 located on each of the one or more supply conduits provide supply pressure greater than the inlet pressure in inlet conduit 130 which opens up the one or more check valves 113, 116 and allows the nutrients to mix with the inlet fluid in the inlet conduit 130. In some embodiments, one or more of the flow control valves 109 and flow modulating valves 110 is configured to isolate the inlet conduit 130 and prevent the flow of nutrients toward the one or more inlet fluid filters 104-107. In some embodiments, the system is configured to deliver (e.g., via one or more pumps 112, 115) the nutrients directly to the one or more bioreactors 118 without mixing with the inlet fluid by isolating the one or more inlet fluid filters 104-107 using one or more of the flow control valves 109 and flow modulating valves 110.

In some embodiments, the system 100 comprises one or more water conditioning assemblies including one or more filters 104-107 and/or one or more sterilization systems 117. In some embodiments, each sterilization system 117 comprises one or more ultraviolet light sources (e.g., LEDs, conventional light sources) configured to kill at least a portion of living microorganisms present in the inlet conduit. In some embodiments, the one or more sterilization systems are positioned between the outlet of each of the one or more nutrient supply conduits 132, 133 and the bioreactor 118. Consideration was given to the inclusion of an ozone generator as a fluid sterilization system, in which case the ultraviolet light would be used to remove ozone from the sterilized water. However, applicant has discovered that sufficient sterilization of the inlet fluid is realized with the application of ultraviolet lights alone according to some embodiments. In some embodiments, ozone can have a detrimental effect on the algae if not removed before introduction into the bioreactor 118. Therefore, in some embodiments, the system 100 does not include an ozone generator. In some embodiments, the system 100 does not employ the use of an ozone generator to sterilize one or more portions of the system.

In some embodiments, the one or more sterilization systems are integrated into the inlet conduit 130 such that the one or more sterilization systems do not comprise a reservoir to store the inlet fluid and/or the nutrient and inlet fluid mixture (hereafter, “the inlet fluid mixture) downstream of the one or more filters 104-107. Advantageously, has been discovered by the inventors that by placing the sterilization system downstream of the one or more nutrient source suppliers 111 the inlet fluid and/or the fluid mixture can be sterilized before being delivered to the one or more bioreactors 118. Therefore, in some embodiments, the sterilization system 117 is not located upstream of the one or more nutrient source suppliers 111 to obtain the benefit of being able to sterilize the fluid mixture. The system 100 is configured and arranged to enable the sterilization system 117 to sterilize both an inlet fluid and a fluid mixture at the same location.

In some embodiments, after the inlet fluid and/or fluid mixture passes the sterilization system 117 it is delivered to the bioreactor 118. In some embodiments, there are no additional reservoirs and/or filters between the sterilization system and the bioreactor 118. In some embodiments, the bioreactor 118 is configured for continuous algae growth propagation. In some embodiments, at least a portion of the bioreactor comprises clear walls for receiving one or more of natural and/or artificial light for continuous microalgae growth propagation. In some embodiments, the bioreactor 118 includes a microculture inlet 120 that may be opened or isolated by a microculture inlet valve 128. In some embodiments, the bioreactor 118 includes a removable top 119 to enable access to the interior of the bioreactor 118 for cleaning and/or maintenance. In some embodiments, the bioreactor may include one or more bioreactor sensors 127 configured to measure one or more of fill level, temperature, pH level, pressure, salinity, flowrate, and/or light intensity. In some embodiments, the bioreactor is configured to receive a gas (e.g., CO₂, O₂, N₂, air, and/or any other desired gas) via a bioreactor pressurized gas supply system. In some embodiments, the pressurized gas supply system includes one or more or a prefilter 121, an air pump 122, a filter 123, and a gas inlet 129. The prefilter 121 is positioned before the air pump 122 to remove damaging particulate from the incoming gas before being drawn into the air pump 122 and subsequently forced through the filter 123 (e.g., a HEPA filter) and into the bioreactor 118 through gas inlet 129.

In some embodiments, the bioreactor 118 includes a bioreactor outlet 135, an outlet isolation valve 124, an outlet pump 125, and an algae outlet 126 that delivers the algae to one or more of an irrigation system (not shown), fluid supply conduit 131 downstream of inlet conduit 130, directly to atmosphere, directly to a location comprising topsoil, and/or any location described herein.

In some embodiments, the algae outlet 126 does not comprise delivery to a recirculation loop that returns any algae to a location previous to the sterilization system 117. In some embodiments, the system 100 is configured to deliver an algae culture to a algae outlet 126 beyond which no further processing is performed on and/or chemicals are added to the algae culture. In some embodiments, the system is not part of a water filtration system where an output from the system includes purified water devoid of algae. In some embodiments, the system does not include any sterilization system or filtration system past (downstream of) the bioreactor.

In some embodiments, the bioreactor 118 is configured to enable the flow of algae out of the bioreactor 118 by displacement caused by inlet fluid and/or a fluid mixture entering the bioreactor. In some embodiments, the system 100 is configured to enable a continuous flow of algae from the bioreactor 118 to the algae outlet 126 when one or more inlet isolation valves 102 are open. In some embodiments, the system 100 is configured to enable a continuous flow of algae from the bioreactor 118 to the algae outlet 126 by displacing algae out of the bioreactor as a result of inlet fluid and/or a fluid mixture entering the bioreactor from the sterilization system 117. In some embodiments, the system 100 is configured to enable a predetermined concentration of algae culture to be maintained within the bioreactor 118 as algae is displaced by establishing the flowrate into the bioreactor such that the mass flowrate of algae out of the system 100 through algae outlet 126 is substantially equal to or less than the growth rate of the microalgae within the bioreactor 118. In some embodiments, the system is configured and arranged to provide contiguous fluid flow through the inlet conduit 130, the one or more filters 104-107, the sterilization system 117, into the bioreactor 118. In some embodiments, the system 100 is configured to enable the contiguous flow to drive the algae out of the bioreactor 118 and to the algae outlet 126.

In some embodiments, the system 100 includes a solar array 200. In some embodiments, the solar array 200 includes one or more of one or more solar panels 201-203, one or more power distributors 204, and one or more battery arrays 205-206. In some embodiments, the solar array 200 is configured to generate, store, and/or supply electrical power to one or more system components.

Some embodiments of the system include a system configured to deliver a full range of micronutrients within microalgae to soil. In some embodiments, the system is configured to inoculate microalgae containing fluid (effluent) directly into soil thereby making the micronutrients immediately bioavailable to crops grown in the soil. In some embodiments, the system is configured to be operatively connected to at least one irrigation system. In some embodiments, the system is configured to be operatively connected between the fluid source and the fluid ports of the at least one irrigation system, through which irrigation fluid is configured to be applied to crops. In some embodiments, the system is configured to produce biofertilizers that are immediately bioavailable to crops, such that negligible runoff pollution occurs. Using this system, inorganic agricultural chemicals can be used more efficiently after being converted or assimilated into a bioavailable form by the algae; therefore, the amount of chemicals needed is reduced according to some embodiments.

In some embodiments, the system is configured to build soil organics with nutrient-rich algae biomass to recover depleted (nutrient poor) soils. In some embodiments, the system is configured to enable and/or accelerate the transformation of a chemicals-based farm to an organic farm. In some embodiments, the system is configured to deliver microalgae to the soil that indirectly dissolve soil carbonates, build polysaccharide content in the topsoil, and improve soil porosity to values that include a range of 500% or more. In some embodiments, the system also includes the use of specific algal biotoxins in place of conventional chemical fungicides and other chemical poisons/toxins to manage nematodes and other harmful pests. In some embodiments, the system only outputs water and microbes from one or more system outlets. In some embodiments, the only chemicals that the system outputs are derived from an algae's organic material, supplied from the algae's organic material, and/or are from a source of nutrient and/or growth propagation for the algae (e.g., microalgae feed).

Some embodiments include a system that comprises one or more bioreactors. In some embodiments, the system comprises a plurality of bioreactors. In some embodiments, when plural bioreactors are present, one or more bioreactors are the same or different. Likewise, in some embodiments, the contents of one or more bioreactor are the same or different. In some embodiments, the bioreactor of the system can comprise one or more types of microalgae. Some embodiments of the system includes microalgae wherein: a) all of the microalgae are of the same type; b) two or more different types of microalgae are present; and/or c) one or more bioreactors contain one or more types of microalgae, and one or more other bioreactors contain one or more other types of microalgae.

In some embodiments, the bioreactor is configured to propagate an initial microalgae inoculant placed into the bioreactor for providing an endless supply of microalgae. In some embodiments, microalgae feed and water can be loaded into the bioreactor and a sufficient amount of microalgae biomass can be removed from the bioreactor continuously and/or periodically so as to keep the conditions within the bioreactor suitable for microalgae culture. In some embodiments, a step of delivering a microalgae biomass to a external holding tank and/or to a topsoil surface includes one or more of removing biomass from the bioreactor, and leaving at least a portion of microalgae within the bioreactor, where the portion left is sufficient to enable continued propagation within the bioreactor. The chemical composition within the bioreactor will vary based on the type of algae in a specific location, but Table 1 is a non-limiting example of the list of mineral components that may be found in an algae culture:

TABLE 1
Example Culture Mixture Mineral Composition

| | 1 × Concentration |
|---|---|
| Components | (g/L) |
| Potassium Nitrate | 166 |
| Magnesium Chloride Hexahydrate | 24.7 |
| Ammonium Nitrate | 22 |
| Potassium Phosphate Monobasic | 10 |
| Calcium Chloride Anhydrous | 10.0 |
| EDTA | 5 |
| Potassium Phosphate Dibasic | 5 |
| Ferric (III) Chloride | 3.187 |
| Citric Acid | 2.0 |
| Manganese Chloride tetrahydrate | 0.18 |
| Zinc Sulfate heptahydrate | 0.11 |
| Cobalt Chloride hexahydrate | 0.02 |
| Sodium Molybdate Dihydrate | 0.0125 |
| Biotin vitamin H B7 | 0.00002 |
| Thiamine HCl | 9.98349E−06 |
| B12 | 1.00026E−06 |
| B6 pyridoxine hydrochloride | 1.21533E−06 |

In some embodiments, the system and its method of use can improve overall crop production within a range of 5% to 30% as compared to untreated crops. In some embodiments, the system and its method of use can improve overall crop production to greater than 30% as compared to untreated crops. In some embodiments, the system and method of use can improve the texture, taste, size, nutrient content and/or yield of a crop as compared to untreated crop. In terms of agriculture use, in some embodiments, the system and its method of use can reduce total energy consumption, and/or reduce ecological pollution, and/or reduce greenhouse gas emission, and/or increase bioavailability of micronutrients and macronutrients, and/or reduce the use of chemical fertilizers, and/or reduce overall crop production cost, and/or reduce tillage cost, and/or reduce the need for and use of fungicides, herbicides and/or pesticides, and/or reduce soil compaction, and/or improve soil porosity, and/or increase microbial content of soil, and/or increase the organics content of soil, and/or reduce the amount of irrigation water needed to grow a crop, and/or reduce the occurrence of over fertilization, and/or reduce run-off and soil erosion, and/or improve plant characteristics and/or improve water/moisture retention by soil, all as compared to untreated crop and croplands.

In some embodiments, the system can be used to reduce or eliminate the buildup of carbonates in irrigation equipment by flowing microalgae-containing water through the irrigation equipment. Some embodiments are directed to a system and method for dissolving carbonates within a system's piping and/or components that includes: (a) providing one or more components of the system described herein; (b) delivering a live microalgae culture to the piping and/or components; (c) containing the microalgae culture within the pipe for a sufficient time to dissolve one or more types of mineral carbonates. In some embodiments, when the algae remain in the piping in the absence of light, they metabolize stored sugars and respire CO2. This becomes carbonic acid in the water and dissolves the calcium carbonate that builds up at the emitters. In some embodiments, the method further includes delivering the algae to topsoil to reduce or eliminate buildup of carbonates in the soil by inoculating the soil with microalgae-containing water.

Many different species and strains of microalgae can be used according to the crop needs. Algae may be collected and cultivated from the field where crops are to be grown or from commercial sources. Microalgae samples can be obtained from repositories at Arizona State University, University of California at Berkeley, University of Texas at Austin, Woods Hole Oceanographic Research Institute, Scripps Institute of Oceanography, or other repositories.

Different species and strains of microalgae grow best under different conditions. The culture conditions within the bioreactor will be varied according to the particular species of microalgae present in the bioreactor. Conditions for culturing many different types of microalgae can be found in The Handbook of Microalgal Culture: Biotechnology and Applied Phycology (ed. Amos Richmond, Blackwell Publishing, Oxford, U. K., 2004), Algal Culturing Techniques: A Book for All Phycologists (ed. Robert A. Andersen, Elsevier Academic Press, 2005), and Microalgae: Biotechnology and Microbiology Cambridge Studies in Biotechnology (ed. E. W. Becker. Press Syndicate of the University of Cambridge, 1994), the disclosures of which are hereby incorporated in their entirety by reference.

In some embodiments, indigenous microalgae species possess properties that make it optimal for growth under the environmental conditions of the target geographic location. In some embodiments, algae from non-indigenous locations or algal collections may be used to inoculate the soil of the target geographic location in order to maximize specific bioavailable compounds. Some embodiments include a method of inoculating soil that can comprise: (a) obtaining a sample of soil from a target geographic location, and/or (b) isolating a robust indigenous microalgae species from the sample, and/or (c) culturing the microalgae to form a first inoculate. Further, in some embodiments, the method includes inoculating a microalgae-based soil inoculating system with the first inoculate, and/or culturing the microalgae in the inoculating system to form a second inoculate, and/or inoculating soil of the target geographic location one or more times with the second inoculate. Further details are disclosed below.

In some embodiments, the system of the system can employ various different types of water as the fluid source, including, but not limited to, wastewater, and/or well water, and/or lake water, and/or creek water, and/or pond water, and/or rainwater, and/or river water and/or freshwater. Since the water is intended for crop growth, it is preferred that the water source has low salinity and is free from heavy metals according to some embodiments. In some embodiments, after exiting the micro-algae inoculating system such as the system of FIG. 1, the inoculate-containing water can be delivered to a crop by any conventional irrigation means or system used in agriculture. In a non-limiting example, in some embodiments, the inoculate containing water is delivered by one or more of a flood, sprinklers, drip type of irrigation systems, and a sprayer or aerial application outlet. If applied by sprayer or aerial application, in some embodiments the treatment method includes a step of supplying sufficient water to drive the algae into the soil.

In some embodiments, the system and methods provide for continuous, semi-continuous, repeated, and/or periodic treatment of soil with a microalgae-containing inoculate. For example, in some embodiments, the soil can be treated with microalgae-containing inoculate daily, or every other day, or every third day, or semi-weekly, or every fourth day, or every fifth day, or every sixth day, or weekly, or biweekly, or every third week, or every fourth week, or monthly, or bimonthly, or quarterly, each trimester, or semiannually, or annually. In some embodiments, the soil can be treated with water not containing the microalgae and then with water containing microalgae inoculate, or vice versa. Some embodiments include a dilute, semi-concentrated and concentrated algal cultures with a single algal species or two or more different algal species. In some embodiments, although it is optional, additional crop nutrients (macronutrients and/or micronutrients), aside from microalgae feed, can be included in the irrigation water. For example, in some embodiments, the nutrients such as zinc may be incorporated into the algal species for transport and uptake by the crops. The following table includes example macronutrients and micronutrients.

| Macronutrients | | Micronutrients | |
|---|---|---|---|
| Nitrogen | (N) | Boron | (B) |
| Phosphorus | (P) | Sulfur | (S) |
| Potassium | (K) | Copper | (Cu) |
| Carbon | (C) | Chloride | (Cl) |
| Oxygen | (O) | Iron | (Fe) |
| Magnesium | (Mg) | Molybdenum | (Mo) |
| Calcium | (Ca) | Manganese | (Mn) |
| | | Nickel | (Ni) |
| | | Zinc | (Zn) |
| | | Selenium | (Se) |
| | | Chromium | (Cr) |
| | | Cobalt | (Co) |
| | | Biotin | |
| | | Thiamin | |
| | | Vitamin B12 | |
| | | Vitamin B6 | |

Algae operate symbiotically with other organisms, both microorganisms and macro-organisms. While the primary object of the system focuses on culturing algae, the system described herein is also configured to culture algae in a diverse community of multiple microorganisms which also offers useful solutions.

Nitrogen-fixing microbes, called diazotrophs, fall into two main groups, free-living and symbiotic. Aerobic diazotrophs, of which there are over 50 genera, including Azotobacter, methane-oxidizing bacteria, and cyanobacteria, require oxygen for growth and fix nitrogen into soil when oxygen is present. Azotobacter, some related bacteria, and some cyanobacteria fix nitrogen in ordinary air, but most members of this group fix nitrogen only when the oxygen concentration is low. Aphanizomenon flosaquae reduces acetylene and fixes nitrogen in algal cultures. Some symbiotic bacteria belong to the genus Rhizobium such as Bradyrhizobium and Sinorhizobium, which colonize the roots of leguminous plants and stimulate the formation of nodules within which they fix nitrogen micro-aerobically. Green microalgae provide nitrogen, phosphorous, potassium, calcium and various other micronutrients. Accordingly, some embodiments include one or more microalgae are co-cultured with or are inoculated into soil along with one or more diazotrophs. In some embodiments, the system includes one or more diazotrophs tanks (not shown) comprising one or more diazotrophs located downstream of the one or more bioreactors 118 and/or connected to the algae outlet conduit 134. In some embodiments, the one or more diazotrophs tanks comprise one or more pumps and or one or more isolation valves (e.g., globe valves, butterfly valves, check valves) similar to the one or more nutrient source suppliers 111, 114 and pump 112, 155, arrangement shown in FIG. 1.

In some embodiments, suitable microorganisms that can be co-cultured with or inoculated into soil along with the microalgae and/or algae can include actinomycetes, bacteria, fungi, and/or mycorrhizae. For example, some embodiments include actinomycetes, which are thread-like bacteria that look like fungi. While not as numerous as bacteria, they perform vital roles in the soil, where they help decompose organic matter into humus, which slowly releases nutrients. They also produce antibiotics to fight root diseases. The same antibiotics can be used to treat human diseases. Actinomycetes create the sweet, earthy smell of biologically active soil when a field is tilled. In some embodiments, the system is configured to deliver one or more suitable microorganisms to a distribution location and/or holding tank through a system outlet together with and/or independently from one or more microalgae inoculants disclosed herein. In some embodiments, the system includes one or more actinomycetes, bacteria, fungi, and/or mycorrhizae tanks (not shown) comprising one or more of actinomycetes, bacteria, fungi, and/or mycorrhizae located downstream of the one or more bioreactors 118 and/or connected to the algae outlet conduit 134. In some embodiments, the one or more actinomycetes, bacteria, fungi, and/or mycorrhizae tanks comprise one or more pumps and or one or more isolation valves (e.g., globe valves, butterfly valves, check valves) similar to the one or more nutrient source suppliers 111, 114 and pump 112, 155, arrangement shown in FIG. 1.

Some embodiments include the use of bacteria which can break down complex molecules and enable plants to take up nutrients. In some embodiments, the system is configured to deliver bacteria to a distribution location and/or holding tank through a system outlet together with and/or independently from one or more microalgae inoculants disclosed herein. Some species release N, S, P and trace elements from organic matter. Others break down soil minerals and release K, P, Mg, Ca and Fe. Other species make and release natural plant growth hormones, which stimulate root growth. A few bacteria fix N in the roots of legumes while others fix N independently of plant association. Bacteria are responsible for converting N from ammonium to nitrate and back again depending on soil conditions. Various bacteria species increase the solubility of nutrients, improve soil structure, fight root diseases, and detoxify soil. In some embodiments, bacteria suitable for co-culture with the microalgae and for use in the system of the system are disclosed in U.S. Pat. No. 7,736,508 to Limcaco (Jun. 15, 2010), the relevant disclosure of which is hereby incorporated by reference. In some embodiments, the system includes one or more bacteria tanks (not shown) comprising one or more types of bacteria located downstream of the one or more bioreactors 118 and/or connected to the algae outlet conduit 134. In some embodiments, the one or more bacteria tanks comprise one or more pumps and or one or more isolation valves (e.g., globe valves, butterfly valves, check valves) similar to the one or more nutrient source suppliers 111, 114 and pump 112, 155, arrangement shown in FIG. 1.

Some embodiments include the use of fungi, some species of which can appear as thread-like colonies, while others are one-celled yeasts. Many fungi aid plants by breaking down organic matter or by releasing nutrients from soil minerals. Fungi are generally early to colonize larger pieces of organic matter and begin the decomposition process. Some fungi produce plant hormones, while others produce antibiotics including penicillin. Several fungi species trap harmful plant-parasitic nematodes. In some embodiments, the system is configured to deliver one or more fungi species to a target surface and/or holding tank through a system outlet together with and/or independently from one or more microalgae inoculants disclosed herein. In some embodiments, the system includes one or more fungi tanks (not shown) comprising one or more types of fungi described herein located downstream of the one or more bioreactors 118 and/or connected to the algae outlet conduit 134. In some embodiments, the one or more fungi tanks comprise one or more pumps and or one or more isolation valves (e.g., globe valves, butterfly valves, check valves) similar to the one or more nutrient source suppliers 111, 114 and pump 112, 155, arrangement shown in FIG. 1.

Some embodiments can include the use of mycorrhizae, a group of fungi that lives either on or in plant roots and act to extend the reach of root hairs into the soil. Mycorrhizae increase the uptake of water and nutrients especially in less fertile soils. Roots colonized by mycorrihizae are less likely to be penetrated by root-feeding nematodes since the pest cannot pierce the thick fungal network. Mycorrhizae also produce hormones and antibiotics, which enhance root growth and provide disease suppression. The fungi benefit from plant association by taking nutrients and carbohydrates from the plant roots where they live.

Aside from revitalization or nutrient supplementation of soil, some embodiments of the system and method can also be used in place of or to reduce the need for conventional herbicides, pesticides, fungicides and nematocides. For example, in some embodiments, the method includes applying, after harvest, an algal species with specially selected toxins to manage nematodes and other soil predators. The algae with toxins are naturally occurring and typically die out after killing the nematodes. While it is possible for algae to mutate, indigenous algae will be far more robust and quickly crowd out any remaining toxic algae. Microalgae suitable for use as pesticides include algae from the genera Nostoc, Scytonema, and Hapalosiphon. In some embodiments, the system is configured to deliver one or more algal species with specially selected toxins and/or phytotoxins to a distribution location and/or holding tank through a system outlet together with and/or independently from one or more microalgae inoculants disclosed herein. Some embodiments can include the use of the system and methods in places such as soil-based farms, parks, hydroponic farms, aquaponics, nurseries, golf-courses, sporting fields, orchards, gardens, zoos and other such places where crops or plants are grown. Some embodiments can include the use of additional phytotoxins obtainable from microbes are described by Duke et al. (“Chemicals from Nature for Weed Management”, Weed Science, (2002) vol. 50, pg. 138-151). Some non-limiting example phytotoxins include actinonin, brefeldin, carbocyclic coformycin, cerulenin cochlioquinone, coronatine, 1,4-cineole, fischerellin, fumosin, fusicoccin, gabaculin, gostatin, grandinol, hydantocidin, leptospermone, phaseolotoxin, phosphinothricin, podophyllotoxin, prehelminthosporol, pyridazocidin, quassinoid, rhizobitoxin, tagetitoxin, sorgoleone syringotoxin, tentoxin, tricolorin A, thiolactomycin and usnic acid.

Some embodiments can include the use of a bioreactor adapted to receive and use natural light. As such, in some embodiments, the bioreactor can be adapted to permit exposure of microalgae to a light source. In some embodiments, the wall of the bioreactor can comprise a light-permeable material to permit exposure of the microalgae to light.

In some embodiments, the system can be run continuously, semi-continuously or in a batch-type operation.

In some embodiments, the system can further comprise one or more monitors or sensors adapted to monitor: a) growing conditions within the bioreactor; and/or b) microalgae cell titer/cell count in the water; and/or c) pH of the water; and/or d) salinity of the water; and/or e) the presence of undesired microbes in the bioreactor; and/or f) water level; and/or g) water pressure; and/or h) level of microalgae nutrients; and/or i) level of solids in the filtered water; and/or j) the level of undesired compounds in the water; and/or k) oxygen, ozone and/or CO₂ content in the water; and/or l) level of nitrogen compounds in the water; and/or m) clarity or opacity of the water; and/or n) level of desired compound(s) in the water; and/or o) water flow-rate; and/or p) weed algae; and/or q) algal predators; and/or) other contaminants.

In some embodiments, the monitor or sensors can be used to control operation of the system, such as by feedback regulation. In some embodiments, a monitor may generate one or more signals to one or more controllers, which control the flow of materials into and/or out of the system. For example, in some embodiments, a microalgae cell titer monitor may send one or more signals to one or more flow controllers that the flow of source water or microalgae-containing water into and/or out of the system. In some embodiments, a pH monitor may send one or more signals to a CO₂ flow controller that controls the amount of, or rate at which, CO₂ is added to the system. In some further embodiments, a water level monitor may send one or more signals to a water flow controller that controls the amount of or rate of water flow into and/or out of the system. In some embodiments, the system includes one or more controllers configured to control one or more components in response to one or more received signals described herein.

In some embodiments, a water pressure monitor may send one or more signals to a water pressure regulator that controls the amount of or rate of water flow into and/or out of the system. In some further embodiments, a clarity monitor may send one or more signals to a water clarity controller that controls the efficiency of filtration of water in the system. In some other embodiments, a nutrient monitor may send one or more signals to a nutrient source flow controller that controls the amount of or rate at which nutrient for the microalgae is added to the system.

In order to grow, plants and microalgae need nutrients such oxygen, carbon, nitrogen, phosphorus, potassium, magnesium, sulfur, boron, copper, chloride, iron, silicon, sodium, manganese, molybdenum, zinc, cobalt, vanadium, bismuth, iodine, water, carbon dioxide, air, and/or others.

The profile of macronutrients and micronutrients provided by the microalgae will depend upon the strain or species of microalgae used. Plants may require a different spectrum of micronutrients and macronutrients during the different stages of the life cycle of the plant. Some embodiments provide a method of growing crops where the macronutrient and micronutrient profile of microalgae is matched with particular phases in the life cycle of a plant. In some embodiments, a field may receive regular nutrient feedings during crop growth and development with different species used depending on the needs of the crop. For example, microalgae A provides a nutrient profile A, microalgae B provides a nutrient profile B, and a target crop requires a nutrient profile A during the early stages of growth and a nutrient profile B ring of the latter stages of growth. In such a situation, the system is configured to inoculate the soil in which the crop is planted first with microalgae A during the early stages of growth of the target crop, inoculate with microalgae B during the latter stages of growth of the target crop.

Some embodiments include a method of producing a crop comprising one or more of the steps of: planting a crop into soil and inoculating the soil with a first microalgae that provides a first nutrient profile, providing one or more portions of the systems and methods described herein; allowing the plant to pass from a first stage of growth into a second stage of growth; and/or inoculating the soil with a second microalgae that provides a different second nutrient profile at the second stage of growth using one or more portions of the systems and methods described herein. In some embodiments, the first nutrient profile will be optimal for plant growth during the first stage, and the second nutrient profile will be optimal for plant growth during the second stage.

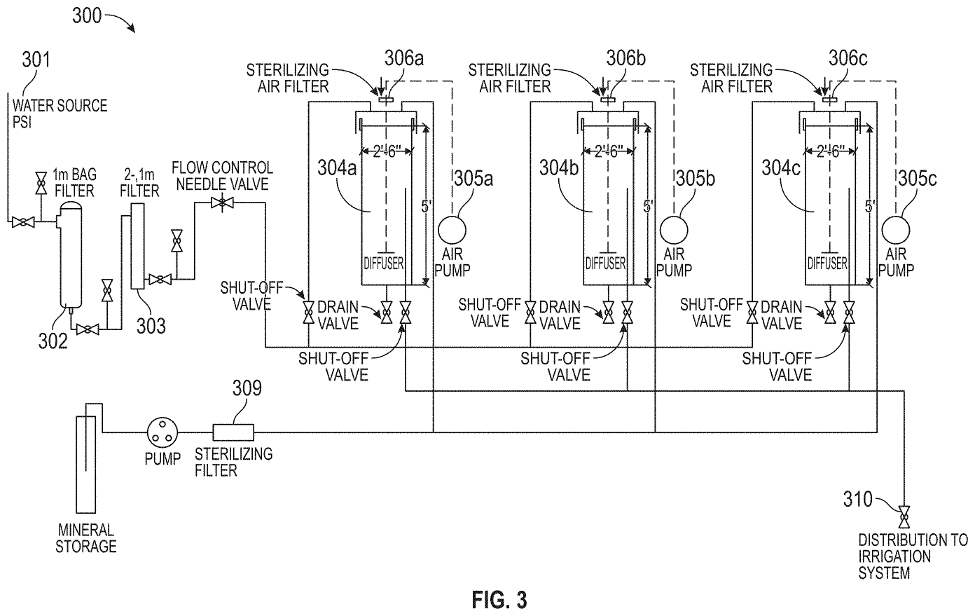

FIG. 3 depicts a microalgae-based soil-inoculating system 300 according to some embodiments. In some embodiments, the system 300 comprises a water source 301, a solids filter 302, a water sterilization filter 303, one or more of bioreactors 304a, 304b, 304c, blowers/air pumps 305a, 305b, 305c, gas sterilization systems 306a, 306b, 306c microalgae nutrient source 307, nutrient pump 308, nutrient sterilization filter 309 and various and water valves and conduits. In some embodiments, gas (e.g., air, CO₂, N₂, O₂) is conducted into the bioreactors 304a-304c and/or into water entering the bioreactors via pumps 305a, 305b, 305c. In some embodiments, the water is filtered through at least one solids filter 302 and at least one fluid sterilization system 303 to form filtered water to which microalgae feed is added by the microalgae nutrient source 307 to form feed water, which is conducted into one or more bioreactors 304a, 304b, 304c. In some embodiments, the sterilization system 303 may include one or more of a mechanical filter and a ultraviolet light source (e.g., UV LEDs). In some embodiments, during initial startup, the bioreactors 304a, 304b, 304c are filled with water containing microalgae nutrients and are then inoculated with a first inoculate containing microalgae. In some embodiments, the gas is injected into the microalgae-containing water in the bioreactors 304a, 304b, 304c using one or more pumps 305a, 305b, 305c. In some embodiments, the microalgae-water in the bioreactors 304a, 304b, 304c is recirculated within the bioreactor for a period of time until the microalgae cell titer/cell count has reached a target level suitable for use as an inoculant. In some embodiments, the water from the system 300 is then flowed into irrigation water system to form a microalgae-containing inoculate as the effluent, which is applied to the soil using the farm irrigation system 310.

In some embodiments, the volume of system water and its flow rate into the irrigation water of the irrigation system 310 is configured to be adjusted as needed to provide the appropriate level of inoculation and water penetration into the soil. For example, in some embodiments, a 200-acre field might receive a total daily volume of 500 to 1 thousand gallons of water at a delivery rate of about 21 gallons/hour to 42 gallons/hour. In some embodiments, the inoculate obtained from the bioreactor (e.g., such as one or more of the bioreactors 304a, 304b, 304c) can be applied to soil with or without further dilution. For example, in some embodiments, the system 301 is configured to be operated such that all water used for irrigation flows through the bioreactor. Otherwise, in some embodiments, the system 1 is configured to be operated such that the inoculate, the effluent of the bioreactors 304a, 304b, 304c, is diluted with additional irrigation water prior to application to the soil.

In some embodiments, the microalgae cell titer (the cell count) in a bioreactor fluctuates over time; therefore, the cell titer of the effluent varies as well. The titer provides important metrics regarding the unit's health and productivity. Generally, the titer in the effluent is configured to be at least 1,000,000 cells per ml up to 30,000,000 cells per ml. The titer is also species specific, and can be higher or lower than the range stated above.

In some embodiments, when a solids filter is present, the system is configured to be used to remove solids from the irrigation water prior to entering the sterilizing filter. In some embodiments, the solids filter can be a flow-through filter. In some embodiments, suitable solids and filters can include the “X100” bag filter from the company filterbag.com or the “FV1” bag filter from the company aquaticeco.com.

In some embodiments, suitable solids filters can include, but not be limited to, media filters, disk filters, screen filters, microporous ceramic filters, carbon-block resin filters, membrane filters, ion-exchange filters, microporous media filters, reverse osmosis filters, slow-sand filter beds, rapid-sand filter beds, cloth filters, and/or any other conventional filter. In some embodiments, the system includes a contiguous train of a plurality of cartridge filters (e.g., four 10″ cartridge filters) with filtration levels of between 50 and 1 micron. The number of filters used in a given application may vary from one to four or more, and the specific filters used in these units may vary depending on water quality.

In some embodiments, a fluid pump can be included in the system. In some embodiments, when present, the fluid pump is configured to facilitate the flow of fluid through the fluid conduits and/or bioreactors of the system. In some embodiments, the system does not include a fluid pump configured to pressurize the inlet conduit with inlet fluid. In some embodiments, if a fluid pump is not included, the pressure of the source fluid (e.g., irrigation water) entering is configured to be sufficient to drive water through the system, including the one or more filters 104-107. Advantageously, some embodiments described herein are configured to operate without the use of a pump, which reduces manufacturing cost and power supply requirements.

In some embodiments, the system includes a pressurized air supply system (e.g., an air pump or blower: both terms are used interchangeably herein). In some embodiments, the pressurized air supply system is configured to facilitate the flow of air through the air conduits, water source and/or bioreactors of the system. In some embodiments, the pressurized air supply system includes a gas pump 305a-305c and gas filters 306a-306c.

The size or operating capacity of each piece of equipment comprising the system can be varied as needed. For example, in some embodiments, a system comprising a total bioreactor capacity of 500 gallons of culture medium can support 200 acres of land and will generally require the following minimum operating capacities for the indicated components: a) solids filter—40 g/min maximum flow with a minimum 2 ft² surface area; d) water pump—10 gal/min minimum; e) air blower/air pump—2.5 cfm at 60″ H₂O minimum; f) microalgae feed source—10×10⁶ cells/ml minimum.

In some embodiments, the system can further comprise one or more monitoring devices (e.g., sensors) for performing functions, including, but not limited to, measuring CO₂ content in the culture, O₂ content in the culture, pH, cell density and temperature in the culture, measuring macronutrient content in the culture or effluent, measuring micronutrient content in the culture or effluent, or measuring the microalgae titer in the culture or effluent. In some embodiments, these can be coupled with a telemetry device and or one or more controllers to allow remote monitoring of the system.

As used herein, a telemetry device can be any device capable of facilitating communication between the system of the system and a communications and/or control center remote from or at a different geographic locale than the system of the system. In some embodiments, the telemetry device is configured to employ any type of wireless communication system and is configured to employ any frequency of light waves, radio waves, sound waves, infrared waves, hypersonic waves, ultraviolet waves, other such wavelengths/frequencies and combinations thereof. In some embodiments, the telemetry device employs an IP network (such as the Internet), GSM (global system for mobile communications) network, SMS (short message service) network, other such systems and combinations thereof.

Some embodiments are configured to be used to reclaim degraded or abandoned soil. In some embodiments, an algae and microorganism mixture produced by the system may be applied though irrigation or spaying on the soil surface to restore vital nutrients. Algae and the other microorganisms continue to flourish in the soil as long as soil moisture is available. Algae deliver micronutrients, attract other microorganisms and add organic matter (humus) to the soil. In some embodiments, the process is configured to rehabilitate degraded or abandoned soil.

In some embodiments of the system, the algae may be delivered through a variety of means including, but not limited to, canal irrigation, flood irrigation, and/or drip irrigation, and/or various conventional overhead spray techniques, and/or various conventional hydroponic cultivation techniques. In some embodiments of the system, the effects of delivering algae to the agricultural production area may be an increase in soil organic matter, and/or improvement in soil structure, and/or reduction in water and fertilizer utilization, and/or increase in crop yield and the nutrient value of the product, and/or an overall improvement in soil health, and/or reduction in water and chemical runoff, and/or an increase in carbon dioxide sequestered from the air by the soil.

Some embodiments of the system include a method of obtaining a soil and/or water sample from an agricultural production area, and/or culturing microbes from the soil sample, and/or selecting a desirable species from the soil sample, and/or propagating the selected desirable species in greater numbers and concentration, and/or delivering live microbes back to the agricultural production area (e.g., such as dispersing the live microbes in solution over a soil area of a farm, or biome area).

In view of the above description and the examples below, one of ordinary skill in the art will be able to practice the system as claimed without undue experimentation. The foregoing will be better understood with reference to the following examples. All references made to these examples are for the purposes of illustration. The following examples should not be considered exhaustive, but merely illustrative of only a few of the many embodiments contemplated by the present system. The examples are understood to constitute one or more methods for using the system in accordance with some embodiments, and one or more actions described in the example methods below constitute one or more method steps employed by the system. Any methods steps from each example, or from any procedure described herein, are understood as being combinable when defining the metes and bounds of the system as a whole.

### Example 1

### Crop Growth Employing Two Different Microalgae

Prior to planting the seeds of a crop in soil, the soil is irrigated repeatedly with an inoculate containing a first species from the phylum Chlorophyta of microalgae until the soil has achieved the desired properties of increased organics with polysaccharides in the soil to increase water retention in accordance with some embodiments. Seeds are planted in the treated soil and irrigated repeatedly with an inoculate containing a different second species from the phylum Cyanophyta of microalgae to infuse the soil with nitrogen sequestered from the atmosphere until the crop has reached maturity in accordance with some embodiments. The crop is then harvested using known methods in accordance with some embodiments. At this point a third species also from the phylum Cyanophyta is introduced into the irrigation water and delivered to the soil where it produces a biological toxin to kill unwanted pests in the soil in accordance with some embodiments. The first species of the phylum Chlorophyta of microalgae is used to enhance the fertility and other properties of the soil by increasing the organics in the soil which enhances the colonization by other micro and macro organisms which further enhance the soil by converting nutrients into forms more available to the crop and by increasing the porosity of the soil in accordance with some embodiments. The second species from the phylum Cyanophyta of microalgae is used to add nitrogen to the soil thereby reducing the amount of nitrogen fertilizer needed by the crop in accordance with some embodiments. The third species from the phylum Cyanophyta is used to eliminate or reduce the number of pests in the soil in accordance with some embodiments.

### Example 2

### System Employing Co-Culture of Two Different Microalgae

A system containing a co-culture of two different microalgae strains are prepared by preparing a culture medium in one or more bioreactors and inoculating it with one or more blue-green algae (cyanobacteria or Cyanophyta) and one or more green algae (Chlorophyta) in accordance with some embodiments. Both algae can be independently unicellular or colonial; however, unicellular, flagilated, mixotrophic species are preferred in accordance with some embodiments. Some Chlorophyta include those of the class Chlorophyceae, which includes those of the order Chaetopeltidales, Chaetophorales, Chlamydomonadales, Chlorococcales, Chlorocystidales, Dunaliella, Microsporales, Oedogoniales, Phaeophilales, Sphaeropleales, Tetrasporales or Volvocales. Some Chlorophyta species include Chlorella fusca, Chlorella zofingiensis, Chlorella spp., Chlorococcum citriforme, Chlorella stigmataphora, Chlorella vulgaris, Chlorella pyrenoidosa and others in accordance with some embodiments. Some Cyanophyta include those of the order Chroococcales, Gloeobaterales, Nostocales, Oscillatoriales, Pseudanabaenales, and Synechococcales in accordance with some embodiments. The algae are co-cultured with natural and/or artificial light in accordance with some embodiments. The titer of algae in the culture medium is allowed to increase to a target level of about 1 MM to 100 MM cells per ml in accordance with some embodiments. The culture medium is discharged from the bioreactor and mixed in with water for irrigation in accordance with some embodiments.

### Example 3

### Alfalfa Yield and Soil Microbial Population Increase with Algae Application

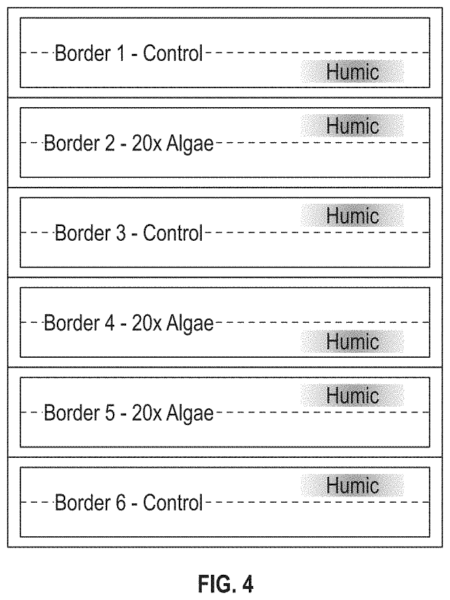

In accordance with some embodiments, a 1.77-acre plot (borders and berms) of agricultural land was tilled, leveled, and had enough berms built to establish six separate borders for alfalfa crop production. Three borders were used as a negative control (no algae applied) and the other three borders were applied with algae during every typical flooding event during the crop cycle in accordance with some embodiments. Half of each of the six borders had a humic product applied and were randomly distributed within the trial field in accordance with some embodiments. The 1.77-acre plot and respective boarders are illustrated in FIG. 4 in accordance with some embodiments.

A system in accordance with some embodiments containing a culture of a microalgae strain was prepared by preparing a culture medium in one or more bioreactors and inoculating it with blue-green algae (cyanobacteria or Cyanophyta) or more green algae (Chlorophyta). Both algae can be independently unicellular or colonial; however, unicellular species are preferred according to some embodiments. Some non-limiting Chlorophyta examples include those of the class Chlorophyceae, which includes those of the order Chaetopeltidales, Chaetophorales, Chlamydomonadales, Chlorococcales, Chlorocystidales, Dunaliella, Microsporales, Oedogoniales, Phaeophilales, Sphaeropleales, Tetrasporales or Volvocales. Some Chlorophyta species include Chlorella fusca, Chlorella zofingiensis, Chlorella spp., Chlorococcum citriforme, Chlorella stigmataphora, Chlorella vulgaris, Chlorella pyrenoidosa and others. Some non-limiting Cyanophyta examples include those of the order Chroococcales, Gloeobaterales, Nostocales, Oscillatoriales, Pseudanabaenales, and Synechococcales. The algae were cultured with natural and/or artificial light in accordance with some embodiments. The titer of algae in the culture medium was allowed to increase to a target level of about 30 MM to 227 MM cells per ml in accordance with some embodiments. The culture medium was discharged from the bioreactor and mixed in with water for irrigation in accordance with some embodiments.

Baseline soil samples were collected to three feet in 1-foot increments with the Giddings soil probe before the first injection of algae in accordance with some embodiments. The final soil samples were also collected to three feet with the Giddings soil probe following one year of algae injections in accordance with some embodiments. The baseline and final samples were analyzed for all the physical, chemical, and biological parameters in accordance with some embodiments. Mid-season samples were surface samples down to 12″ in accordance with some embodiments. Soil samples were collected from the half where the humics are applied and the half without in accordance with some embodiments. Physical measurements were made on all soil samples in accordance with some embodiments. The elemental chemical analyses were made on only the baseline and final soil samples from the non-humic halves collected at 1′ in accordance with some embodiments. Biological parameters were measured on the 1′ baseline, in-season and final samples from the non-humic halves in accordance with some embodiments. In total, 12 samples while will have the chemical elemental analyses—six from baseline and six from final, and 18 samples will have the biological tests (i.e., PLFA and Haney) —six from baseline, mid-season, and final in accordance with some embodiments.

Estimated yields were calculated by harvesting a pre-determined square footage of biomass equal to all six of the borders and then measuring both the wet weight and dry weight of the biomass in grams in accordance with some embodiments. The estimated dry weight tonnage yield per acre is extrapolated by multiplying the dried biomass weight and the size of each border in accordance with some embodiments.

After the first cut, both the averages of the algae treatments and the algae+humic product treatments exhibited a dry weight increase of over 30% in comparison to the averages of the negative control treatment in accordance with some embodiments. During the biomass harvest, soil samples were taken at the same time to determine the population size and percent distribution of microbial populations in the soil in accordance with some embodiments. Both the averages of the algae treatments and the algae+humic product treatments exhibited a total living microbial biomass increase of 129% and 190% respectively in comparison to the averages of the negative control treatment which only had a 76% increase in total living microbial biomass in accordance with some embodiments.

The subject matter described herein is directed to technological improvements to the field of soil rejuvenation by providing systems and methods for the delivery of a culture of one or more microbes from a specific location to the same location. The disclosure describes the specifics of how a machine including one or more controllers and/or one or more computers comprising one or more processors and one or more non-transitory computer implement the system and its improvements over the prior art. The instructions executed by the machine cannot be performed in the human mind or derived by a human using a pin and paper but require the machine to convert process input data to useful output data. Moreover, the claims presented herein do not attempt to tie-up a judicial exception with known conventional steps implemented by a general-purpose computer; nor do they attempt to tie-up a judicial exception by simply linking it to a technological field. Indeed, the systems and methods described herein were unknown and/or not present in the public domain at the time of filing, and they provide a technologic improvements advantages not known in the prior art. Furthermore, the system includes unconventional steps that confine the claim to a useful application.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein when describing the metes and bounds of the system as a whole.

Some embodiments of the system are presented with specific values and/or setpoints. These values and setpoints are not intended to be limiting and are merely examples of a higher configuration versus a lower configuration and are intended as an aid for those of ordinary skill to make and use the system.

Furthermore, acting as Applicant's own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured.

“Simultaneously” as used herein includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry. “Simultaneously” also includes actuation delays in mechanical systems.

As used herein, “can” or “may” or derivations there of (e.g., the system display can show X) are used for descriptive purposes only and is understood to be synonymous and/or interchangeable with “configured to” (e.g., the computer is configured to execute instructions X) when defining the metes and bounds of the system.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited. Another example is “a computer system configured to or programmed to execute a series of instructions X, Y, and Z.” In this example, the instructions must be present on a non-transitory computer readable medium such that the computer system is “configured to” and/or “programmed to” execute the recited instructions: “configure to” and/or “programmed to” excludes art teaching computer systems with non-transitory computer readable media merely “capable of” having the recited instructions stored thereon but have no teachings of the instructions X, Y, and Z programmed and stored thereon. The recitation “configured to” can also be interpreted as synonymous with operatively connected when used in conjunction with physical structures.

Applicant expressly, clearly and unmistakably disavows any system where the intended purpose of the system is to provide an output and/or recirculation loop that is devoid of an algae culture and/or living biological organisms at the system outlet (e.g., water purification systems). In addition, Applicant expressly, clearly and unmistakably disavows any systems that prevent a flow of an algae culture from a bioreactor under normal operating when fluid is being continuously supplied to the bioreactor. Furthermore, Applicant expressly, clearly and unmistakably disavows any system that includes a sterilization system configured to kill living organisms downstream a bioreactor outlet. Such art is outside the scope of this disclosure which is expressly directed to systems where the output includes an algae culture. Such a express, clear and unmistakable disavowal of scope for applications in the United States is authorized by and in accordance with MPEP § 2111.01(IV)(B).

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The previous detailed description is to be read with reference to the figures, in which like elements in different figures have like reference numerals. The figures, which are not necessarily to scale, depict some embodiments and are not intended to limit the scope of embodiments of the system.

Any of the operations described herein that form part of the invention are useful machine operations. The invention also relates to a device or an apparatus for performing these operations. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g. a cloud of computing resources.

Although method operations are presented in a specific order according to some embodiments, the execution of those steps do not necessarily occur in the order listed unless a explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

We claim:

1. A system for improving nutrients in topsoil comprising:
one or more filters,
one or more sterilization systems, and
one or more bioreactors;
wherein the one or more filters are configured to filter particulate from inlet fluid supplied from an inlet fluid source;
wherein the one or more sterilization systems are configured to kill living organisms within the inlet fluid; and
wherein the system is configured to supply the inlet fluid to the bioreactor after the inlet fluid has passed through both the one or more filters and the one or more sterilization systems.

2. The system of claim 1,
wherein the one or more bioreactors each comprise at least one bioreactor outlet; and
wherein the system is configured to enable a continuous flow of algae from the at least one bioreactor outlet as a result of the inlet fluid being supplied to the one or more bioreactors.

3. The system of claim 1,
further comprising an algae outlet configured to deliver algae cultured in the one or more bioreactors to outside the system;
wherein the algae outlet is located downstream of the one or more bioreactors; and
wherein the one or more sterilization systems are not located between the at least one bioreactor outlet and the algae outlet.

4. The system of claim 1,
wherein the one or more sterilization systems are not located downstream of the one or more bioreactors.

5. The system of claim 1,
wherein the one or more bioreactors comprises a bioreactor outlet; and
wherein the one or more bioreactors are configured to not prevent algae from exiting the bioreactor.

6. The system of claim 1,
wherein the system does not comprise an ozone generator.

7. The system of claim 1,
wherein the sterilation system comprises one or more ultraviolet light sources.

8. The system of claim 1,
wherein the sterilation system consist of one or more ultraviolet light source.

9. A system for improving nutrients in topsoil comprising:
a fluid source inlet,
a fluid source outlet,
an inlet conduit,
one or more filters,
one or more sterilization systems, and
one or more bioreactors;
wherein the fluid source inlet is configured and arranged to supply pressurized fluid to the fluid source outlet and the inlet conduit;
wherein the inlet conduit comprises the one or more filters and the one or more sterilization systems;
wherein the inlet conduit is configured to supply inlet fluid from the inlet fluid source to the one or more bioreactors;
wherein the one or more filters are configured to filter particulate from inlet fluid supplied from the fluid source inlet;
wherein the one or more sterilization systems are configured to kill living organisms within the inlet fluid supplied from an inlet fluid source; and
wherein the system is configured to supply the inlet fluid to the bioreactor after the inlet fluid has passed through both the one or more filters and the one or more sterilization systems.

10. The system of claim 9,
further comprising:
one or more nutrient source suppliers, and
one or more nutrient supply conduits;
wherein the one or more nutrient source supply conduits are fluidly connected to the inlet conduit and the one or more nutrient source suppliers; and
wherein the one or more nutrient source supply conduits are configured to supply nutrients from the one or more nutrient source suppliers to the inlet conduit.

11. The system of claim 10,
wherein the one or more nutrient source supply conduits are fluidly connected to the inlet conduit upstream of the one or more bioreactors.

12. The system of claim 11,
wherein the inlet conduit does not comprise a pump.

13. The system of claim 10,
wherein the one or more nutrient source supply conduits are fluidly connected to the inlet conduit between the one or more filters and the sterilation system.

14. The system of claim 10,
wherein the sterilization system is configured to kill at least a portion of living organisms in the nutrients and the inlet fluid simultaneously.

15. The system of claim 10,
wherein the one or more nutrient supply conduits are configured to prevent the inlet fluid from flowing into the one or more one or more nutrient source suppliers.

16. The system of claim 11,
wherein the one or more bioreactors each comprise at least one bioreactor outlet; and
wherein the system is configured to enable a continuous flow of algae from the at least one bioreactor outlet as a result of the inlet fluid being supplied to the one or more bioreactors.
